(19) United States
(12) Patent Application Publication
(10) Pub. No.: US 2026/0041909 A1
(43) Pub. Date: Feb. 12, 2026

(54) ELECTRODE ARRAY AND ELECTRODE FOR PERIPHERAL NERVE STIMULATION

(71) Applicant: AURIMOD GMBH, Wien (AT)

(72) Inventor: Stefan KAMPUSCH, Wien (AT)

(73) Assignee: AURIMOD GMBH, Wien (AT)

(21) Appl. No.: 18/853,688

(22) PCT Filed: Mar. 30, 2023
(86) PCT No.: PCT/IB2023/053189

§ 371 Date: Apr. 28, 2025

(30) Foreign Application Priority Data
Apr. 4, 2022 (EP) 22020150.3

(51) Int. Cl. A61N 1/05; A61N 1/04; A61N 1/36

(52) U.S. Cl. CPC A61N 1/0558; A61N 1/0476; A61N 1/0502; A61N 1/36017; A61N 1/36034

(57) ABSTRACT

An electrode array for peripheral nerve stimulation, in particular for auricular punctual stimulation of the vagus nerve, including a planar carrier and a plurality of needle-shaped electrodes protruding from the carrier and at least one electrical terminal, which is electrically conductively connected to the electrodes or to a subgroup of electrodes, for electrical connection to a stimulation device, the electrodes having a maximum length of 0.6 mm, preferably 0.4 mm

### CROSS-REFERENCE TO RELATED APPLICATIONS

The present application is a national phase application of PCT Application No. PCT/IB2023/053189, filed Mar. 30, 2023, entitled “ELECTRODE ARRAY AND ELECTRODE FOR PERIPHERAL NERVE STIMULATION”, which claims the benefit of European Patent Application No. 22020150.3, filed Apr. 4, 2022, each of which is incorporated by reference in its entirety.

### BACKGROUND OF THE INVENTION

### 1. Field of the Invention

The invention relates to an electrode array, electrode, or a device for peripheral nerve stimulation, in particular for auricular punctual stimulation, e.g. of the vagus nerve. In particular, the device comprises a current generator for generating stimulation current pulses and at least one electrode array or at least one electrode which is connected to the current generator by means of an electrical line.

### 2. Description of the Related Art

Available stimulation devices for the electrical stimulation of the auricular vagus nerve are used in the therapy of a wide variety of conditions, such as chronic and acute pain, epilepsy and depression. In auricular punctual stimulation devices, as disclosed, for example, in WO 2011/030210 A1, electrical stimulation pulses are introduced via needle electrodes, which are inserted into the skin at specified points of the auricle and remain there for the treatment period of, for example, several days.

Stimulation makes it possible, for example, to advantageously influence pain processing and pain perception. Stimulation also positively influences the sympathovagal balance in the autonomic nervous system. As a result, the patient's current physiological state changes dynamically, measured as changes in heart rate, respiratory rate, blood pressure, local blood flow, and other parameters.

Stimulation devices for peripheral nerve stimulation mostly use surface electrodes. These are, for example, ball electrodes that rest against the skin or are pressed against the skin by mechanical fit, e.g. with the shape of the auricle. In the field of application of transcutaneous nerve stimulation, there are also adhesive electrodes, which are usually glued to the area of skin to be stimulated over a large area. The stimulation thus takes place over a larger area of skin, since the electrodes must be of a certain size in order not to cause burns, since high stimulation energies must be used to overcome the skin resistance. Affixing the electrodes, apart from adhesive electrodes, is often difficult here and usually limits the application to intermittent, non-chronic use. Another disadvantage of large-area surface electrodes lies in the locally unspecific stimulation.

On the other hand, there are applications of percutaneous stimulation, where small needle electrodes are inserted up to 2 mm deep into the skin to excite nerve fibers. With such needle electrodes, one can stimulate very specifically and overcome the skin resistance with the resulting lower stimulation energy compared to surface electrodes, since the skin resistance is no longer relevant. Here, too, affixing is not easy, but easier than with surface electrodes, which in principle allows chronic use over several days. However, longer needle electrodes hurt when inserted and can sometimes be difficult to fix for a longer period of time. The mostly elastic tissue pushes the needle electrodes out of the tissue again over time.

In principle, efforts are made to reduce the stimulation energy as much as possible in order to achieve a miniaturized design and a high energy efficiency of the stimulation devices used.

### SUMMARY OF THE INVENTION

The present invention therefore aims to overcome the above-mentioned disadvantages and provide an electrode assembly for peripheral nerve stimulation that allows for locally specific stimulation, enables long-term treatment, increases wearing comfort, and minimizes the required stimulation energy. Furthermore, the invention aims to improve an electrode array or a single electrode in such a way that its hold in the tissue is improved and its resistance to being pulled out or falling out is increased.

To achieve this object, the invention according to a first aspect consists of providing an electrode array for peripheral nerve stimulation, in particular for auricular punctual stimulation, e.g. of the vagus nerve, comprising a planar carrier and a plurality of needle-shaped electrodes protruding from the carrier and at least one electrical terminal, which is electrically conductively connected to the electrodes or a subgroup of electrodes, for electrical connection to a stimulation device, the electrodes having a maximum length of 0.6 mm, preferably 0.4-0.6 mm.

The invention is thus based on the approach of using, instead of a single long needle or a surface electrode, an electrode array that has a plurality of short electrodes that penetrate into the tissue, but to a limited extent. The electrodes have a maximum length of 0.6 mm, preferably 0.4-0.6 mm, the length of the electrodes being understood to be the distance between the needle tip and the surface of the planar carrier defining the skin contact surface. The electrode length can therefore be equated with the maximum penetration depth of the electrodes into the skin of a patient.

Thus, on the one hand, the stimulation pulses can be introduced in a location-specific manner and, on the other hand, the stimulation energy can be kept low. Furthermore, the invasiveness is reduced compared to long needle electrodes. As a result, the structure height can be greatly reduced and thus the mechanical stability increased. Furthermore, several small needle electrodes lead to a better fit in the tissue. Pain when puncturing is almost completely prevented. The wearing comfort is significantly increased. For chronic use over several days, it is much easier to affix the electrodes.

Due to the lower stimulation energy and the increased surface area due to multiple needles, the risk of exceeding the current density (“charge injection limit”) at the electrode below which the stimulation pulses do not produce any reaction products and thus do not allow any corrosion processes to take place at the electrode during operation is also reduced.

With regard to the arrangement of the electrodes on the planar carrier, no restrictions are provided in principle. According to a preferred embodiment of the invention, however, the stimulation energy is distributed as uniformly as possible over the region of the tissue covered by the electrode array by arranging the electrodes in a regular grid on the carrier.

The electrodes are preferably arranged at a distance of 0.5-1 mm from one another, the shortest distance between two adjacent electrodes being used here in each case.

The electrode array may comprise at least three electrodes. The electrode array preferably comprises at least 9, preferably at least 16, more preferably at least 25 electrodes.

With regard to the shape of the electrodes, it is preferably provided that they are pointed needle electrodes, which ensures simple and as painless as possible penetration of the skin surface. In particular, the electrodes may be conical, wherein the conical shape may be provided only at the electrode tip or the electrode corresponds to the conical shape over its entire length.

According to a preferred embodiment, an optimized electrode shape is obtained in that the electrodes have, an in particular cylindrical thicker section in a central region, which is adjoined by a conical tip. The thicker section provided in the central region increases the pull-out force, so that the electrode array is held more firmly and stably in the tissue overall.

A further improvement is achieved when the thicker section merges continuously in the direction of the carrier into an in particular cylindrical base section, the diameter of which is smaller than the diameter of the thicker section. This preferred aspect of the invention is not limited to an electrode array, but also relates to the fit of a single electrode. The fact that the thicker section merges continuously into the in particular cylindrical base section, means that the transition from the thicker section to the cylindrical base section is formed without an abrupt change in the diameter of the electrode. The transition from the thicker section to the cylindrical base section has, in particular, a continuously decreasing diameter. The continuous transition ensures that the insertion of the electrode according to the invention into the tissue and the removal of the electrode can take place more or less painlessly.

The aforementioned optimized electrode shape forms an independent, second aspect of the invention, according to which an electrode for peripheral nerve stimulation, in particular for auricular punctual stimulation, e.g. of the vagus nerve, has a thicker section in a central region, which is adjoined by a conical tip. In this case, the electrode preferably has an in particular cylindrical base section, whose diameter is smaller than that of the thickened central region. This second aspect of the invention is not limited to an electrode array, but relates in particular to a single electrode.

Furthermore, it can preferably be provided that the needle tip of the electrodes has a diameter of 0.02-0.04 mm. This results in correspondingly sharp tips that can easily penetrate the skin.

The electrode array according to the invention can be produced from a wide variety of materials. According to a first alternative embodiment, the electrode array consists essentially entirely of an electrically conductive material, in particular of a metal, such as titanium or platinum-iridium, or of a conductive plastic, such as poly-3,4-ethylenedioxythiophene.

According to a second alternative embodiment, at least the electrodes consist of an electrically conductive material, such as titanium or platinum-iridium, or of a conductive plastic, such as poly-3,4-ethylenedioxythiophene, and the carrier consists of an electrically non-conductive material, in particular a polymer.

According to a further alternative embodiment, the electrode array may consist of an electrically non-conductive material and the electrodes are provided with an electrically conductive coating, e.g. of gold or titanium. The coating may be applied by any coating method, such as sputtering or electroplating.

The electrode array may be produced by milling from a solid body, by a casting process, such as injection molding, by punching from a plate or by an additive manufacturing process (3D printing). Additive manufacturing may be carried out, for example, by means of a stereolithographic printing process, whereby a high spatial resolution can be achieved and the electrodes can be produced correspondingly precisely, true to form and with a smooth surface. The electrode array may be printed, for example, from a conductive plastic or from a metallic and therefore electrically conductive material. Alternatively, the electrode array may be printed from a non-conductive material and then coated with a conductive material.

The plate-like carrier of the electrode array according to the invention may be designed as a rigid carrier or as a flexible structure, such as a film or sheet. Alternatively, the carrier may be designed as a conductive gel pad (e.g. conductive hydrogel), which is pierced with the electrode needles and thus forms a flexible electrode. The design as a flexible structure allows the carrier to be easily adapted to the respective surface contour of that part of the body to which the electrode array is to be applied. In the case of a rigid carrier, the carrier may provide a skin contact surface from which the electrodes protrude, which is flat. Alternatively, the skin contact surface of the carrier may be shape-matched to the desired body location.

To improve the attachment of the electrode array to the skin of a patient, the skin contact surface of the carrier may be provided with an adhesive coating.

Several of the electrode arrays according to the invention, each of which is connected to its own stimulation channel of an associated stimulation device, may be used to stimulate the peripheral nerve fibers at a specific body site of a patient. Alternatively, the electrode array according to the invention may also be shaped and used as a larger-area matrix that supports several stimulation channels. In this way, bipolar and mono-, bi-or multi-phase stimulation can be achieved.

In the case of a larger-area electrode array that supports two or more stimulation channels, a preferred embodiment of the invention provides for the electrodes to have at least a first and a second group of electrodes, each group of electrodes being electrically conductively connected to its own electrical terminal.

According to a further aspect of the invention, a device for peripheral nerve stimulation, in particular for auricular punctual stimulation, e.g. of the vagus nerve of a patient, is provided, comprising a current generator for generating stimulation current pulses and at least one electrode array according to the first aspect of the invention or at least two electrodes according to the second aspect of the invention, which is or are each connected to the current generator by at least one electrical line.

For peripheral nerve stimulation, it is particularly advantageous if the current generator is built to generate the stimulation current pulses with a pulse frequency of <1 kHz.

With regard to the current intensity, a preferred embodiment provides that the current generator is built to generate the stimulation current pulses with a current amplitude of >5 mA. The stimulation current pulses may preferably have an alternating polarity.

### BRIEF DESCRIPTION OF THE DRAWINGS

The invention is explained in more detail below with reference to exemplary embodiments schematically illustrated in the drawing.

FIG. 1 shows a schematic representation of a device according to the invention,

FIG. 2 shows the arrangement of needle electrodes on a human ear with a representation of voltage and current for vagus nerve stimulation,

FIG. 3 shows a schematic representation of an electrode array according to the invention,

FIG. 4 shows a view of an exemplary form of an electrode,

FIG. 5 shows a schematic representation of an electrode array with an electrical terminal for a stimulation current line,

FIG. 6 shows an alternative embodiment of an electrode array,

FIG. 7 shows a detailed representation of an electrode attachment,

FIG. 8 shows an alternative embodiment of the electrode attachment,

FIG. 9 shows a further alternative embodiment of an electrode array,

FIG. 10 shows a section along the line A-A of FIG. 9,

FIG. 11 shows a further alternative embodiment of an electrode array, and

FIG. 12 shows a detailed representation of an electrode of the electrode array of FIG. 11.

### DETAILED DESCRIPTION

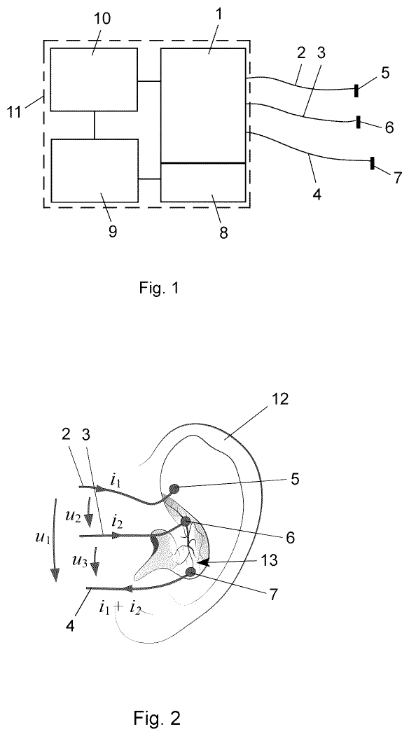

FIG. 1 shows an exemplary embodiment of the device according to the invention, comprising a current or voltage generator 1, which is powered by a battery (not shown) and to which electrical lines 2, 3 and 4 are connected, at each of whose ends an electrode array 5, 6 and 7 to be positioned on the ear is arranged. The generator 1 is built to generate stimulation current pulses, which are introduced into the ear, for example, via the lines 2, 3, 4 and the associated electrode arrays 5, 6, 7. A measuring circuit 8 is provided for measuring the tissue impedance tapped, for example, via the electrode arrays 6 and 7.

Connected to the measuring circuit 8 is a signal processing circuit 9, to which the measured values of the measuring circuit 8 are supplied and which is built to determine at least one physiological measured value, such as the heart rate, heart rate variability, blood flow, vascular stiffness and/or respiratory rate, from the temporal profile of the tissue impedance. Furthermore, a control circuit 10 is provided, which interacts with the current generator 1 to change at least one stimulation current parameter, such as the pulse frequency and/or the current amplitude of the stimulation current pulses. In this case, the at least one stimulation current parameter can be changed as a function of the physiological measured value determined by the signal processing circuit 9 or of its time profile, for which purpose the measured value is supplied to the control circuit 10 by the signal processing circuit 9.

The current generator 1, the measuring circuit 8, the signal processing circuit 9, and the control circuit 10 are arranged in a housing 11 that can be mounted near the ear, e.g., behind the ear. The current generator 1, the measuring circuit 8, the signal processing circuit 9, and the control circuit 10 may be designed as separate units or may be implemented in a common electronic circuit.

FIG. 2 shows the human ear 12 with blood vessels and the afferent vagus nerve branches 13. In the region of the vagus nerve branches 13, the electrode arrays 5, 6 and 7 are inserted into the tissue, a sequence of stimulation pulses with the current i₁ being applied via the line 2 and a sequence of stimulation pulses with the current i₂ being applied via the line 3, and the current reflux i₁+i₂ taking place via the line 4. A voltage u₁ results between lines 2 and 4, a voltage u₂ results between lines 2 and 3, and a voltage u₃ results between lines 3 and 4. Alternatively, a constant voltage can also be impressed and the current measured.

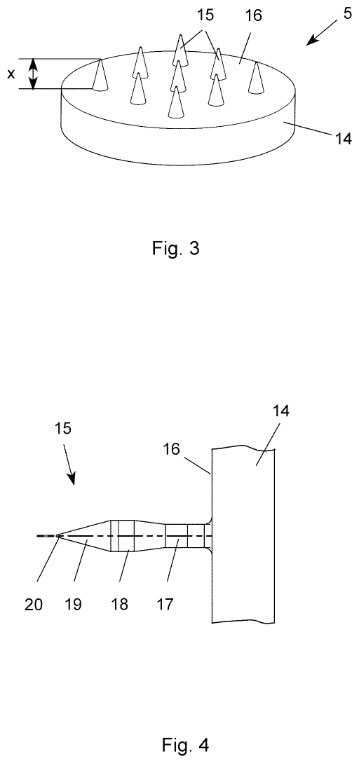

FIG. 3 shows an electrode array 5, wherein the electrode arrays 6 and 7 may be similarly constructed. The electrode array 5 comprises a planar carrier 14, on which a plurality of needle electrodes 15 are arranged in a grid-like manner. The needle electrodes 15 have a length x measured from the skin-facing surface 16 of the carrier 14 of, for example, 0.2-0.6 mm and penetrate the skin surface of a patient. In the embodiment shown in FIG. 3, the needle electrodes 15 are conical.

FIG. 4 shows an alternative shape of a needle electrode 15, which forms an independent subject matter of the present invention. The needle electrode may be used either as part of an electrode array 5 or as a single needle. The needle electrode 15 comprises an in particular cylindrical base section 17, a thickened central region 18, and a conically tapered needle portion 19 with a needle tip 20.

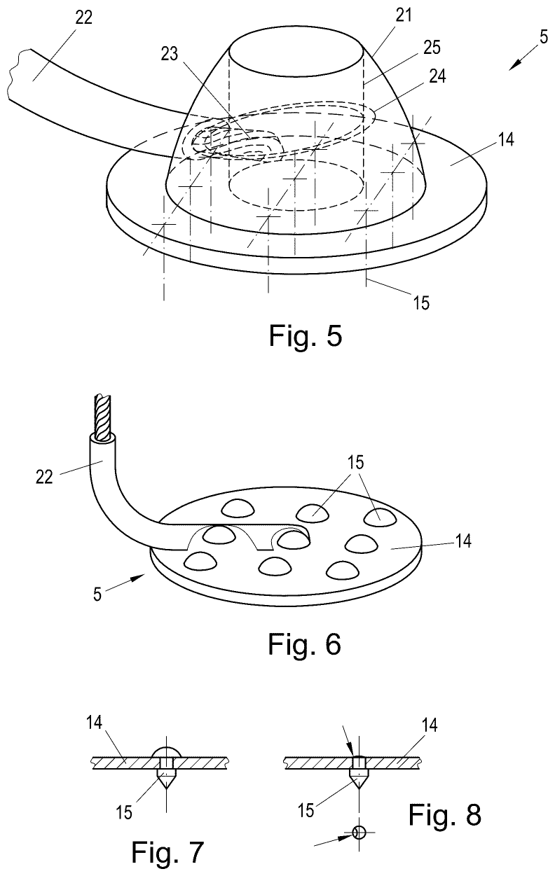

FIG. 5 shows an electrode array 5 of the type shown in FIG. 3 with a carrier 14 and only schematically indicated needle electrodes 15. On the side of the carrier 14 facing away from the needle electrodes 15, a line terminal is provided, which serves for the electrically conductive connection of a stripped end 23 of a current conductor 22 to the electrode array 5. For this purpose, the line terminal has an in particular cylindrical terminal part 25, which is arranged on the carrier 14. To electrically connect the end 23 to the terminal part 25, an annular clamping part 24 is attached to the end 23, for example by soldering or by injection molding the clamping part consisting of conductive plastic. The clamping part is pushed over the cylindrical terminal part 25 and fastened, for example, by a latching or clamping connection, as a result of which an electrically conductive connection is established between the end 23 or the clamping part 24 and the terminal part 25. The line terminal can then be provided with a cover, sheath or encapsulation 21, which can be produced, for example, from a plastic by extrusion coating.

In the alternative embodiment according to FIG. 6, the current conductor 22 is connected to the electrode array 5 by fastening to one of the needle electrodes 15. The fastening can be effected, for example, by clamping, riveting or welding, depending on how the individual needle electrodes 15 are fastened to the carrier 14. In the embodiment according to FIG. 7, the needle electrodes 15 are riveted to the carrier 14. In this case, the current conductor 22 can be clamped between the thickened head of the needle electrode 15 and the carrier 14. In the embodiment according to FIG. 8, the needle electrodes 15 are welded to the carrier 14. In this case, the current conductor 22 can be connected to the needle electrode 15 or the carrier 14 by means of a welding spot.

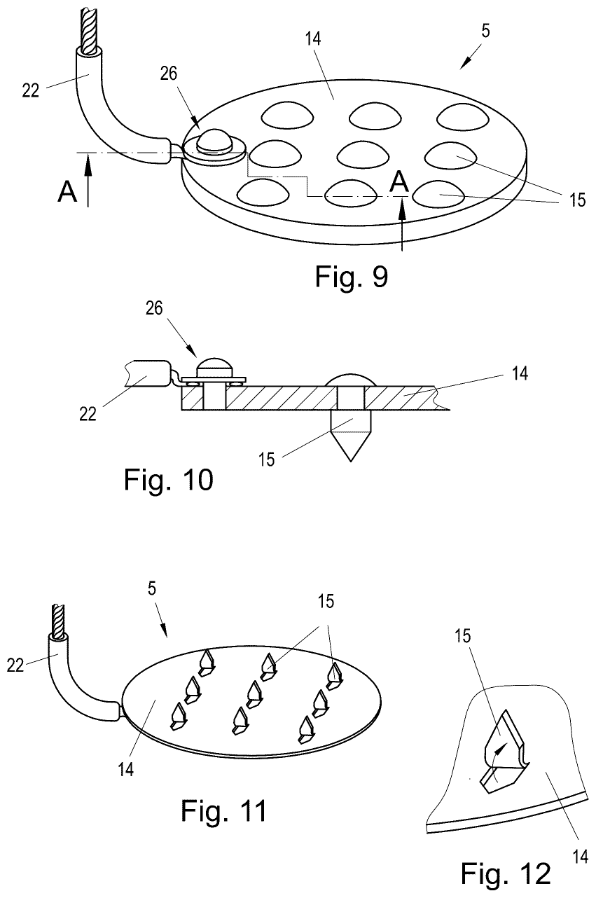

In the alternative embodiment shown in FIGS. 9 and 10, the current conductor 22 is electrically conductively connected to the carrier 14 at its own fastening point 26. In this case, the fastening can be carried out, for example, by means of a clamping pin.

FIGS. 11 and 12 show an alternative embodiment of the electrode array 5, in which the needle electrodes 15 are formed integrally with the carrier 14 and have been bent out of the planar carrier 14 by punching. In this case, the current conductor 22 can also be embossed directly during the punching process.

1-17. (canceled)

18. An electrode array for peripheral nerve stimulation, comprising:
a planar carrier;
a plurality of needle-shaped electrodes protruding from the carrier; and
at least one electrical terminal electrically conductively connected to the electrodes or to a subgroup of electrodes, for electrical connection to a stimulation device;
wherein the electrodes have a maximum length of 0.6 mm;
wherein the electrodes comprise in a central region a thicker section adjoined by a conical tip, the thicker section merging continuously in a direction of the planar carrier into a base section, a first diameter of the base section being smaller than a second diameter of the thicker section.

19. The electrode array according to claim 18, wherein the electrodes have a maximum length of 0.4-0.6 mm.

20. The electrode array according to claim 18, wherein the thicker section is cylindrical.

21. The electrode array according to claim 18, wherein the base section is cylindrical.

22. The electrode array according to claim 18, wherein the electrodes are arranged in a regular grid on the carrier.

23. The electrode array according to claim 18, wherein the electrodes are arranged at a distance of 0.5-1 mm from one another.

24. The electrode array according to claim 18, wherein the conical tip of the electrodes has a diameter of 0.02-0.04 mm.

25. The electrode array according to claim 18, wherein the electrodes have at least a first and a second group of electrodes, each group of electrodes being electrically conductively connected to its own electrical terminal.

26. The electrode array according to claim 18, wherein the electrode array consists of an electrically conductive material or of a conductive plastic.

27. The electrode array according to claim 18, wherein the electrodes consist of an electrically conductive material or of a conductive plastic, and the carrier consists of an electrically non-conductive material.

28. The electrode array according to claim 18, wherein the electrodes are titanium or platinum-iridium.

29. The electrode array according to claim 18, wherein the electrodes are poly-3,4-ethylenedioxythiophene.

30. The electrode array according to claim 18, wherein the carrier consists of a polymer.

31. The electrode array according to claim 18, wherein the electrode array consists of an electrically non-conductive material and the electrodes are provided with an electrically conductive coating.

32. The electrode array according to claim 18, wherein the carrier is built as a flexible film or sheet metal.

33. An electrode for peripheral nerve stimulation, comprising:
a thicker section in a central region; and
a conical tip adjoining the thicker section;
wherein the electrode has a base section, a first diameter of the base section being smaller than a second diameter of the thicker section; and
wherein the thicker section merges continuously into the base section.

34. The electrode according to claim 33, wherein the thicker section is cylindrical.

35. The electrode according to claim 33, wherein the the base section is cylindrical.

36. A device for peripheral nerve stimulation, comprising:
a current generator for generating stimulation current pulses; and
one of:
at least one electrode array for peripheral nerve stimulation, including:
a planar carrier;
a plurality of needle-shaped electrodes protruding from the carrier; and
at least one electrical terminal electrically conductively connected to the electrodes or to a subgroup of electrodes, for electrical connection to a stimulation device;
wherein the electrodes have a maximum length of 0.6 mm;
wherein the electrodes comprise in a central region a thicker section adjoined by a conical tip, the thicker section merging continuously in a direction of the planar carrier into a base section, a first diameter of the base section being smaller than a second diameter of the thicker section; and
wherein the at least one electrode array is connected to a current generator by at least one electrical line; and
at least two electrodes for peripheral nerve stimulation, each of the at least two electrodes including:
a thicker section in a central region; and
a conical tip adjoining the thicker section;
wherein the electrode has a base section, a first diameter of the base section being smaller than a second diameter of the thicker section;
wherein the thicker section merges continuously into the base section; and
wherein the at least two electrodes are each connected to the current generator by the at least one electrical line.

37. The device according to claim 36, wherein the current generator is configured to generate the stimulation current pulses with a pulse frequency of <1 kHz and with a current amplitude of >5 mA.
